(19) United States
(12) Patent Application Publication
(10) Pub. No.: US 2010/0016937 A1
(43) Pub. Date: Jan. 21, 2010

(54) Twisting Bifurcation Delivery System

(76) Inventor: Yousef Alkhatib, Maple Grove, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 12/175,958

(22) Filed: Jul. 18, 2008

(51) Int. Cl. A61F 2/06; A61M 29/00; A61M 25/00

(52) U.S. Cl. 623/1.11; 604/96.01; 600/585

(57) ABSTRACT

A catheter assembly includes a catheter shaft having a proximal portion and a distal portion, the catheter shaft having a torsionally weakened region at the distal portion, the torsionally weakened region including one or more portions that are recessed from an outer surface of the catheter shaft. The assembly also includes a primary guidewire lumen defined in the catheter shaft and sized to receive a primary guidewire, and an inflatable member positioned at the distal portion of the catheter shaft.

### CROSS-REFERENCE TO RELATED APPLICATIONS

This application is related to U.S. patent application Ser. No. 11/272,886 filed on Nov. 14, 2005, the entirety of which is hereby incorporated by reference.

### STATEMENT REGARDING FEDERALLY SPONSORED RESEARCH

Not Applicable.

### BACKGROUND

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. Stents may be used to reinforce body vessels and to prevent restenosis following angioplasty in the vascular system. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Stents may be delivered using suitable delivery systems. For example, a stent may be oriented about an inflation balloon of a delivery catheter. The catheter may be maneuvered through a bodily vessel to deliver the stent to a deployment site. The stent may be expanded by inflating the balloon with an inflation medium, such as a pressurized fluid. The balloon may then be deflated, and the catheter removed from the body.

Within the vasculature, it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

### SUMMARY

The illustrated examples disclosed herein relate generally to catheter assemblies and related methods for treatment of a vessel bifurcation. The catheter assembly includes features that improve alignment of the catheter assembly with an ostium of a branch vessel of the vessel bifurcation. An example catheter assembly includes a main catheter branch configured to reside within the main vessel, and a side catheter branch configured to extend from the main vessel into the branch vessel.

In example arrangements, a catheter assembly includes a catheter shaft having a proximal portion and a distal portion, the catheter shaft having a torsionally weakened region at the distal portion, the torsionally weakened region including one or more portions that are recessed from an outer surface of the catheter shaft. The assembly also includes a primary guidewire lumen defined in the catheter shaft and sized to receive a primary guide wire, and an inflatable member positioned at the distal portion of the catheter shaft.

There is no requirement that an arrangement include all features characterized herein to obtain some advantage according to this disclosure.

### DESCRIPTION OF THE DRAWINGS

A detailed description is hereafter described with specific reference being made to the drawings.

FIG. 1 is a side view of a catheter system with a catheter shaft including a torsionally weakened region.

FIG. 2 is an enlarged view of a portion of the catheter system shown in FIG. 1.

FIG. 3 is a side view of a torsionally weakened region of a catheter shaft of another catheter system.

FIG. 4 is a side view of a torsionally weakened region of a catheter shaft of another catheter system.

FIG. 5 is a side view of a portion of another catheter system with a catheter shaft including a torsionally weakened region and an inner shaft shown exploded therefrom including an inner torsionally weakened region.

FIG. 6 is a side view of a portion of another catheter system with a catheter shaft including a torsionally weakened region and an inner shaft shown exploded therefrom including an inner torsionally weakened region.

FIG. 7 is a side view of another catheter system with a catheter shaft including a torsionally weakened region.

### DETAILED DESCRIPTION

This disclosure relates to bifurcation treatment systems, catheter assemblies, and related methods of treating bifurcations in a patient's body. The term bifurcation means a division location from one unit into two or more units. Generally, two types of bifurcations of a body organ include: 1) a main tubular member defining a main lumen and a branch tubular member defining a branch lumen that extends or branches off from the main tubular member, wherein the main and branch lumens are in fluid communication with each other; and 2) a primary or main member defining a primary or main lumen (also referred to as a parent lumen) that splits into first and second branch members defining first and second branch lumens. The term lumen means the cavity or bore of a tubular structure such as a tubular organ (e.g., a blood vessel).

An example bifurcation is a vessel bifurcation that includes a continuous main vessel and a branch vessel, wherein the vessels define a main lumen and a branch lumen, respectively, that are in fluid communication with each other. Alternatively, a vessel bifurcation can include a parent vessel that divides into first and second branch vessels, wherein the vessels define a parent lumen and first and second branch lumens, respectively, which lumens are all in fluid communication with each other.

Example applications of the principles disclosed herein include cardiac, coronary, renal, peripheral vascular, gastrointestinal, pulmonary, urinary, and neurovascular systems. The catheter assemblies, systems and methods disclosed herein can be used for locating a branch vessel of the vessel bifurcation and for placement of a stent relative to the vessel bifurcation for treatment of the vessel bifurcation.

A wide variety of stents, catheters, and guidewire configurations can be used with the catheter assembly embodiments of the present disclosure. The principles disclosed herein should not be limited to any particular design or configuration. Some example stents that can be used with the catheter assemblies disclosed herein can be found in, for example, U.S. Pat. Nos. 6,210,429, 6,325,826 and 6,706,062 to Vardi et al., co-pending U.S. patent application Ser. No. 10/644,550, filed on Aug. 21, 2003, and titled STENT WITH A PROTRUDING BRANCH PORTION FOR BIFURCATED VESSELS, and U.S. Published Patent Application No. 2004/0176837 titled SELF-EXPANDING STENT AND CATHETER ASSEMBLY AND METHOD FOR TREATING BIFURCATIONS, the entire contents of which are incorporated herein by reference. In general, the aforementioned stents include a lateral branch opening located between distal and proximal open ends of the stent. The lateral branch opening defines a path between an inner lumen of the stent and an area outside of the stent. The stent lateral branch opening is distinct from the cell openings defined between strut structures from which the stent sidewall is constructed. In some stents, the lateral branch opening can be surrounded by expandable structure. The expandable structure can be configured to extend radially into the branch lumen of the bifurcation upon expansion of, for example, an inflatable portion of the bifurcation treatment system. Typically, the stent is expanded after being positioned in the main lumen with the lateral branch opening aligned with an opening into the branch lumen. Alignment of the lateral branch opening with the opening into the branch lumen includes both radial and axial alignment. The stent, including the expandable structure surrounding the lateral branch opening, can be expanded with a single expansion or multiple expansions using one or more inflatable members.

The main and side balloons, and all other balloons disclosed herein, can be made of any suitable balloon material including compliant and non-compliant materials and combinations thereof. Some example materials for the balloons and catheters disclosed herein include thermoplastic polymers, polyethylene (high density, low density, intermediate density, linear low density), various co-polymers and blends of polyethylene, ionomers, polyesters, polycarbonates, polyamides, poly-vinyl chloride, acrylonitrile-butadiene-styrene copolymers, polyether-polyester copolymers, and polyetherpolyamide copolymers. One suitable material is Surlyn®, a copolymer polyolefin material (DuPont de Nemours, Wilmington, Del.). Still further suitable materials include thermoplastic polymers and thermoset polymeric materials, poly(ethylene terephthalate) (commonly referred to as PET), thermoplastic polyamide, polyphenylene sulfides, polypropylene. Some other example materials include polyurethanes and block copolymers, such as polyamide-polyether block copolymers or amide-tetramethylene glycol copolymers. Additional examples include the PEBAX® (a polyamide/polyether/polyester block copolymer) family of polymers, e.g., PEBAX® 70D, 72D, 2533, 5533, 6333, 7033, or 7233 (available from Elf AtoChem, Philadelphia, Pa.). Other examples include nylons, such as aliphatic nylons, for example, Vestamid L21011F, Nylon 11 (Elf Atochem), Nylon 6 (Allied Signal), Nylon 6/10 (BASF), Nylon 6/12 (Ashley Polymers), or Nylon 12. Additional examples of nylons include aromatic nylons, such as Grivory (EMS) and Nylon MXD-6. Other nylons and/or combinations of nylons can also be used. Still further examples include polybutylene terephthalate (PBT), such as CELANEX® (available from Ticona, Summit, N.J.), polyester/ether block copolymers such as ARNITEL® (available from DSM, Erionspilla, Ind.), e.g., ARNITEL® EM740, aromatic amides such as Trogamid (PA6-3-T, Degussa), and thermoplastic elastomers such as HYTREL® (Dupont de Nemours, Wilmington, Del.). In some embodiments, the PEBAX®, HYTREL®, and ARNITEL® materials have a Shore D hardness of about 45 D to about 82 D. The balloon materials can be used pure or as blends. For example, a blend may include a PBT and one or more PBT thermoplastic elastomers, such as RITEFLEX® (available from Ticona), ARNITEL®, or HYTREL®, or polyethylene terephthalate (PET) and a thermoplastic elastomer, such as a PBT thermoplastic elastomer. Additional examples of balloon material can be found in U.S. Pat. No. 6,146,356, which is incorporated herein by reference.

Aligning a stent with the lateral branch opening of a vessel bifurcation is a difficult task and is achieved by allowing the catheter system to rotate into place via guidewires. A branch guidewire and a main guidewire are positioned within the branch vessel and in the main vessel in order to define a path by which a catheter assembly can track to the vessel bifurcation. As the catheter assembly tracks along the two guidewires, the catheter assembly will twist and/or rotate as a result of the torque created by tracking along the guidewires.

Because a catheter shaft of the catheter assembly needs to be able to twist under low torque loads, the catheter shaft may be constructed to include a flexible portion that facilitates twisting and/or rotation of the catheter assembly. Various materials and constructions can be used to provide the flexibility needed to achieve adequate rotation and/or twisting. By making the catheter shaft more flexible in some regions than in other regions, the catheter system may be advanced through the vasculature with less difficulty, thereby potentially reducing damage to the vessel walls as well as increasing delivery accuracy.

It should be noted that the flexible portion can be embodied in fixed wire devices, over-the-wire devices, rapid exchange devices, MONORAIL® devices, as well as other devices that are known and used by others of ordinary skill in the art of bifurcation treatment.

In some arrangements, a stent being delivered by the catheter assembly to the bifurcation treatment site is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

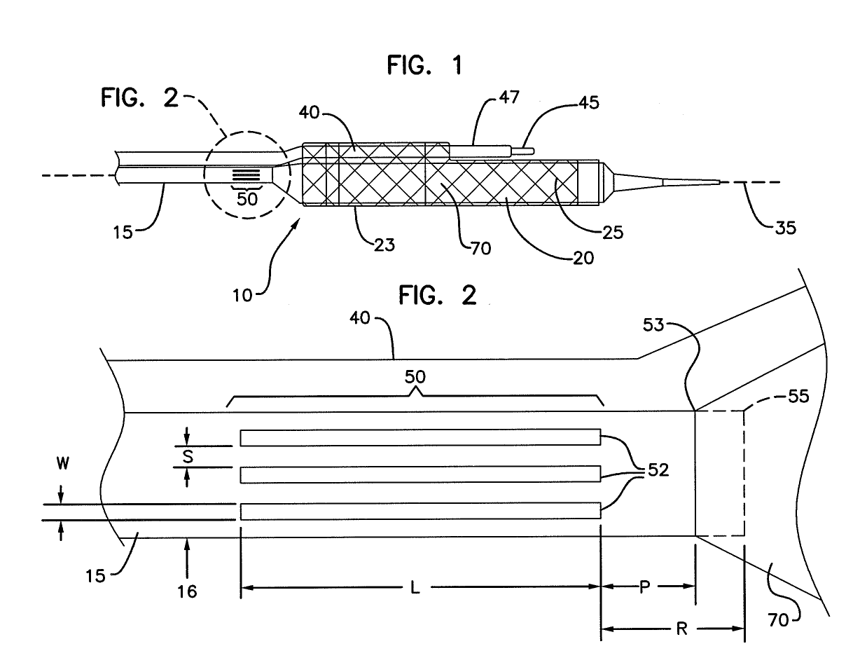

Referring now to FIGS. 1 and 2, an example catheter system 10 is shown. Catheter system 10 is comprised of a catheter shaft 15, expandable balloon 70, side branch guidewire housing 40, and side branch guidewire 45 which extends through side branch guidewire lumen 47. The distal region 20 of catheter shaft 15 defines a stent retaining region 25 for stent 23.

Catheter shaft 15 of catheter system 10 includes a region 50 located adjacent to expandable balloon 70. Generally, region 50 is a torsionally weakened region that is configured to enhance the flexing or twisting of catheter shaft 15 about a longitudinal axis 35 relative to balloon 70. Region 50 is flexible, in that region 50 more readily twists when torque is applied as compared to the other portions of catheter shaft 15.

As noted above, changing the flexibility characteristics of catheter shaft 15 using region 50 allows catheter system 10 to be advanced through the vasculature with less difficulty and increases delivery accuracy.

In the illustrated arrangement, region 50 includes a plurality of longitudinal portions 52 formed therein. Generally, each portion 52 is recessed from an outer surface 16 of catheter shaft 15, such that each portion 52 defines an area in which a portion of catheter shaft 15 has been removed or is void. Each portion 52 includes a length L, a width W, and a depth (not shown). Region 50 is located at a distance P relative to a proximal end 53 of balloon 70, and a distance R relative to a distal end 55 of catheter shaft 55.

The overall surface area of portions 52 (i.e., the surface area defined by the length and width dimensions for the portions 52), as well as the relative position of region 50 on catheter shaft 15, can be varied to increase or decrease the flexibility of catheter shaft 15. In some embodiments, the total surface area of the portions 52 is compared to the total surface area of the circumference of the catheter shaft 15 to determine the number of regions 50 and/or portions 52 within each region 50 to be used.

For example, if a greater number of portions 52 is added to region 50, the flexibility of shaft 15 is increased and an amount of torque required to twist catheter shaft 15 about longitudinal axis 35 is thereby decreased. Likewise, if the number of portions 52 is decreased, or length L, width W, or the depth is decreased, the flexibility of shaft 15 is decreased accordingly.

A spacing S between adjacent portions 52 can also be varied as desired. In the examples shown, spacing S can be decreased to increase the flexibility of region 50, or vice versa. Other configurations are possible.

Dimensions for portions 52 can be of uniform length L, width W, depth, and spacing S, or the dimensions can be varied from one portion to another. For example, in one alternative, length L of every other portion 52 is decreased to decrease the flexibility of region 50. In yet another embodiment, the depth of some of portions 52 is increased to thereby increase the flexibility of region 50. Other alternatives are possible.

In examples, portions 52 can be positioned about the entire circumference of catheter shaft 15 to form region 50. In other arrangements, portions 52 can be positioned about only a portion of the circumference of catheter shaft 15 to form region 50.

In the example shown, region 50 is positioned adjacent to balloon 70. In other arrangements, region 50 can be positioned at alternative positions on catheter shaft 15. For example, distance P between region 50 and proximal end 53 of balloon 70 can be decreased to increase the twisting characteristics of catheter shaft 15 relative to balloon 70. In yet other designs, multiple regions 50 can be included along catheter shaft 15 to further increase the flexibility of shaft 15 to increase the twisting characteristics thereof.

Portions 52 can be formed in catheter shaft 15 in a variety of manners. In one arrangement, portions 52 are formed using an ultraviolet (“UV”) laser ablation process, wherein longitudinal portions of material from catheter shaft 15 are removed to form region 50. For example, UV laser ablation is used to remove longitudinal portions of material that extend parallel to one another and parallel to longitudinal axis 35 of catheter shaft 15 to form portions 52.

In alternative arrangements, portions 52 can be formed using other processes. For example, portions 52 can be formed during the manufacture of catheter shaft 15, such as during the molding process for catheter shaft 15. In yet another arrangement, portions 52 can be formed by removing portions of catheter shaft 15 using a sharp object or objects.

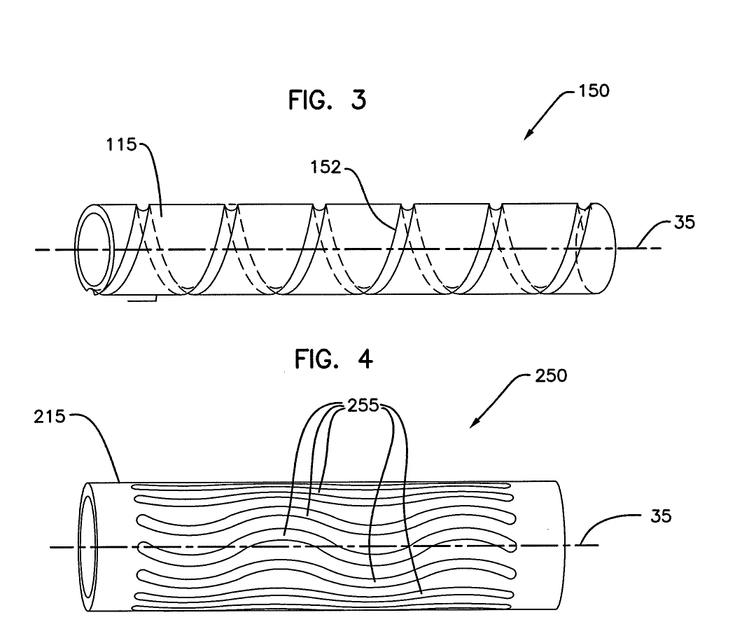

Referring now to FIGS. 3 and 4, alternative designs for the torsionally weakened region of a catheter shaft 115 are shown. In FIG. 3, a region 150 of catheter shaft 115 includes a helical or angled portion 152 formed thereon that winds helically about longitudinal axis 35. In FIG. 4, a region 250 of a catheter shaft 215 includes a plurality of undulating or wavy portions 252 extending along longitudinally axis 35. Other configurations are possible. The width, length, depth, and/or spacing of portions 152 and 252 can be varied to change the twisting characteristics of regions 150 and 250, respectively.

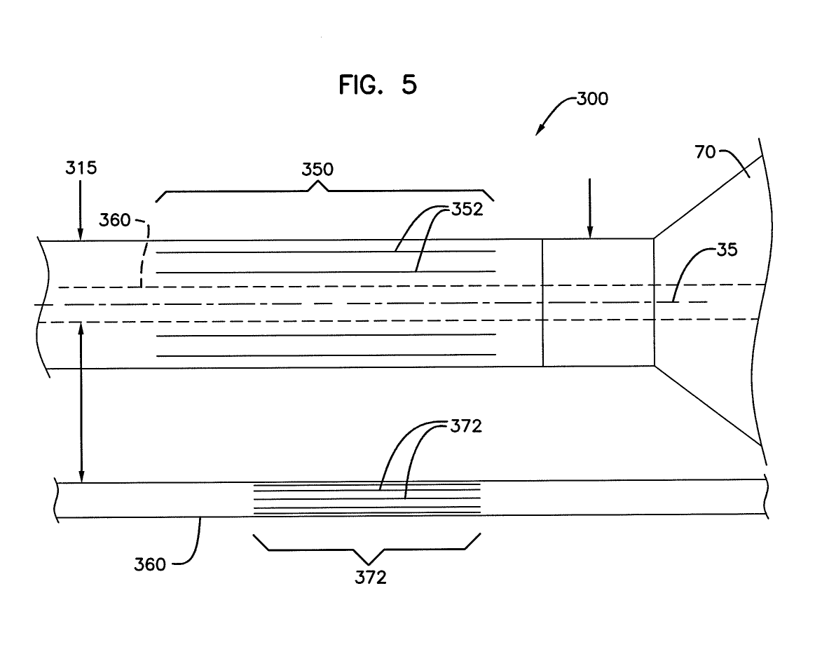

Referring now to FIG. 5, a portion of another example catheter system 300 is shown. A catheter shaft 315 of catheter system 300 includes a region 350 located adjacent to expandable balloon 70. Region 350 includes a plurality of portions 352 extending parallel to one another and parallel to longitudinal axis 35 of catheter shaft 315.

An inner shaft 360 extends along longitudinal axis 35 within catheter shaft 315. Inner shaft 360 includes a region 370 with a plurality of portions 372 formed therein in a manner similar to portions 352 of catheter shaft 315. In the example shown, region 370 of inner shaft 360 is positioned within catheter shaft 315 adjacent to region 350 to further enhance the twisting characteristics thereof. In other examples, region 370 can be spaced longitudinally from region 350.

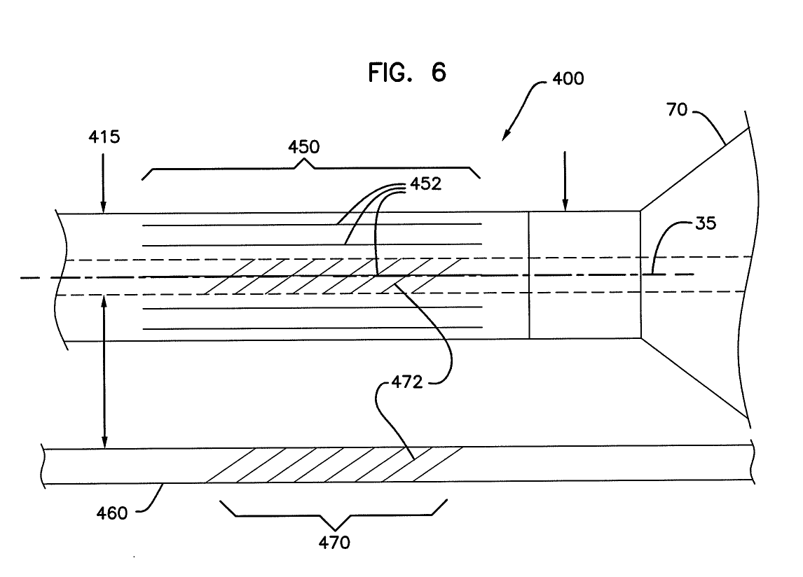

Referring now to FIG. 6, another example catheter system 400 is shown. A catheter shaft 415 of catheter system 400 includes a region 450 having a plurality of portions 452 extending parallel to one another and parallel to longitudinal axis 35 of catheter shaft 415. An inner shaft 460 extending along longitudinal axis 35 within catheter shaft 415 includes a region 470 with a portion 472 formed therein. Portion 472 winds helically about inner shaft 460 along longitudinal axis 35. Region 470 of inner shaft 460 is positioned within catheter shaft 415 adjacent to region 450 to further enhance the twisting characteristics thereof.

In alternative examples, other configurations for the portions formed on the outer or inner shafts of the catheter systems can be used. For example, in another arrangement, only the inner shaft includes portions. In another example, one or both of the outer and inner shafts include portions running in a plurality of directions.

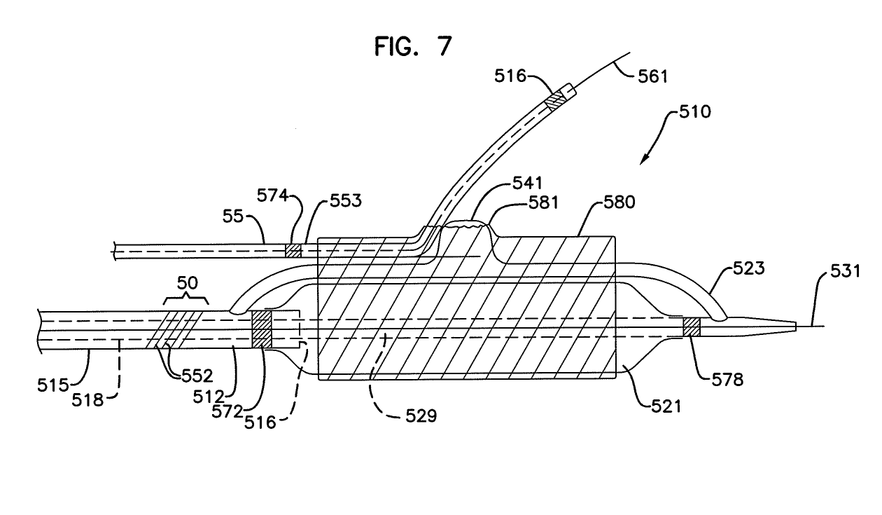

Referring now to FIG. 7, another example catheter system 510 is shown. Catheter system 510 includes a catheter shaft 515 including an inner shaft 518 extending therein. Inner shaft 518 defines a first lumen 529 through which a guidewire 531 extends. A distal end 516 of catheter shaft 515 is coupled to a primary inflatable member 521.

An inflation lumen 512 extends within catheter shaft 515 to inflatable member 521. A secondary inflation lumen 532 extends to a side inflatable member 541 located adjacent to inflatable member 521. In the example shown, inflation lumen 512 and secondary inflation lumen 532 are in fluid communication within catheter shaft 515 at a position proximal to or downstream from a branching of lumens 512, 532, so that both inflatable members 521, 541 can be inflated concurrently. Other arrangements are possible.

Catheter system 510 also includes a secondary guidewire housing 551 defining a secondary lumen 553 for a secondary guidewire 561 to pass therethrough. In example embodiments, a stent 580 is positioned about inflatable members 521, 541 and secondary guidewire housing 551. Secondary guidewire housing 551 extends through a lateral branch opening 581 in stent 580 so that secondary guidewire housing 551 can be aligned with an ostium of a branch vessel of a vessel bifurcation during deployment.

In the example shown, catheter shaft 515 includes a torsionally weakened region 550 located adjacent to distal end 516 of catheter shaft 515 and inflatable member 521. Torsionally weakened region 550 has a plurality of portions 552 formed therein to enhance the flexibility of catheter shaft 515 at torsionally weakened region 550. For example, torsionally weakened region 550 can be configured with portions 552 such that torsionally weakened region 550 promotes twisting such that catheter shaft 515 twists more readily when torque is applied during the delivery of inflatable members 521, 541 and stent 580. In this manner, torsionally weakened region 550 can twisted more easily to locate secondary guidewire housing 551 within a bifurcated vessel as inflatable members 521, 541 are located at a desired position with a vessel.

Catheter system 510 can include marker material that is visible under X-ray or in fluoroscopy procedures. For example, markers 572, 574, 576, 578 are positioned along the distal end portions of catheter shaft 515 and secondary guidewire housing 551. Any features of the system 510 that include marker material can be more easily identified and distinguished under X-ray or in fluoroscopy procedures. Some example marker materials include gold, platinum and tungsten. In one embodiment, the marker material can be included in a band structure that is secured to at least one of catheter shaft 515 and secondary guidewire housing 551. In other embodiments, the marker material is part of the material composition of portions of catheter shaft 515 and secondary guidewire housing 551. Viewability of features of the catheter system 510 under X-ray or fluoroscopy can assist the physician operating the system 510 to more easily adjust a position of the system 510 relative to the vessel bifurcation. Example markers and marker materials suitable for use with system 510 are described in U.S. Pat. No. 6,692,483 to Vardi, et al., and co-pending U.S. provisional patent application Ser. No. 60/776,149, filed on Feb. 22, 2006, and titled MARKER ARRANGEMENT FOR BIFURCATION CATHETER, which patent matters are incorporated herein by reference.

In one embodiment, a catheter assembly includes a catheter shaft having a proximal portion and a distal portion, the catheter shaft having a torsionally weakened region at the distal portion, the torsionally weakened region including one or more portions that are recessed from an outer surface of the catheter shaft. The assembly also includes a primary guidewire lumen defined in the catheter shaft and sized to receive a primary guidewire, and an inflatable member positioned at the distal portion of the catheter shaft.

In another embodiment, a catheter system for deployment in a bifurcated vessel including a catheter shaft including a proximal portion and a distance portion and extending along a longitudinal axis, an inflatable member coupled to a distal portion of the catheter shaft, and a torsionally weakened region of the catheter shaft being positioned adjacent to the inflatable member, the torsionally weakened region including a plurality of portions that are recessed from an outer surface of the catheter shaft such that the torsionally weakened region increases twisting of the distal portion of the catheter shaft when torque is applied thereto. The system also includes a primary guidewire lumen positioned in the catheter shaft, the primary guidewire lumen being sized to receive a primary guidewire, and a side branch guidewire housing coupled to the catheter shaft, the side branch guidewire housing being sized to receive a secondary guidewire.

In another embodiment, a method of forming a catheter shaft includes: forming the shaft extending along a longitudinal axis from a proximal end to a distal end, the shaft defining a guidewire lumen therein; forming an inflatable member coupled to the distal end of the shaft; and ablating the shaft adjacent to the inflatable member to form a plurality of portions that are recessed with respect to an outer circumference of the shaft to form a torsionally weakened region.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to.”

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from an independent claim should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

What is claimed is:

1. A catheter assembly, comprising:
(a) a catheter shaft having a proximal portion and a distal portion, the catheter shaft having a torsionally weakened region at the distal portion, the torsionally weakened region including one or more portions that are recessed from an outer surface of the catheter shaft;
(b) a primary guidewire lumen defined in the catheter shaft and sized to receive a primary guidewire; and
(c) an inflatable member positioned at the distal portion of the catheter shaft.

2. The catheter of claim 1, wherein the portions are parallel portions that are formed about the torsionally weakened region and extend along the longitudinal axis.

3. The catheter of claim 2, wherein the portions are formed by ablating the catheter shaft.

4. The catheter of claim 1, wherein the portions are helical portions that are formed about the torsionally weakened region.

5. The catheter of claim 1, wherein the portions are formed by ablating the catheter shaft.

6. The catheter of claim 1, further comprising an inner shaft positioned within the catheter shaft, the inner shaft including an inner torsionally weakened region positioned adjacent to the torsionally weakened region of the catheter shaft.

7. The catheter of claim 1, further comprising a side branch guidewire housing coupled to the catheter shaft, the side branch guidewire housing being sized to receive a secondary guidewire.

8. A catheter system for deployment in a bifurcated vessel, the system comprising:
(a) a catheter shaft including a proximal portion and a distance portion and extending along a longitudinal axis;
(b) an inflatable member coupled to a distal portion of the catheter shaft;
(c) a torsionally weakened region of the catheter shaft being positioned adjacent to the inflatable member, the torsionally weakened region including a plurality of portions that are recessed from an outer surface of the catheter shaft such that the torsionally weakened region increases twisting of the distal portion of the catheter shaft when torque is applied thereto;
(d) a primary guidewire lumen positioned in the catheter shaft, the primary guidewire lumen being sized to receive a primary guidewire; and
(e) a side branch guidewire housing coupled to the catheter shaft, the side branch guidewire housing being sized to receive a secondary guidewire.

9. The system of claim 8, wherein the portions are parallel portions that are formed about the torsionally weakened region and extend along the longitudinal axis

10. The system of claim 8, wherein the portions are helical portions that are formed about the torsionally weakened region.

11. The system of claim 8, wherein the portions are formed by ablating the catheter shaft.

12. The system of claim 8, further comprising:
(f) an inner shaft positioned within the catheter shaft, the inner shaft including an inner torsionally weakened region positioned adjacent to the torsionally weakened region of the catheter shaft.

13. The system of claim 12, wherein the inner torsionally weakened region is defined by a plurality of inner portions formed in the inner shaft.

14. The system of claim 13, wherein the plurality of inner portions are parallel inner portions.

15. The system of claim 13, wherein the plurality of inner portions are formed by ablating the inner shaft.

16. A method of forming a catheter shaft, the method comprising:
(a) forming the shaft extending along a longitudinal axis from a proximal end to a distal end, the shaft defining a guidewire lumen therein;
(b) forming an inflatable member coupled to the distal end of the shaft; and
(c) ablating the shaft adjacent to the inflatable member to form a plurality of portions that are recessed with respect to an outer circumference of the shaft to form a torsionally weakened region.

17. The method of claim 16, wherein ablating the shaft further comprises forming parallel portions about the shaft that extend along the longitudinal axis.

18. The method of claim 16, wherein ablating the shaft further comprises forming helical portions about the shaft that extend along the longitudinal axis.

19. The method of claim 16, further comprising:
(d) forming an inner shaft extending within the shaft along the longitudinal axis; and
(e) forming a plurality of inner portions in the inner shaft adjacent to the plurality of portions on the shaft.

20. The method of claim 16, further comprising:
(d) coupling a side branch guidewire housing to the shaft, the side branch guidewire housing being sized to receive a secondary guidewire.
